(19) United States
(12) Patent Application Publication
(10) Pub. No.: US 2011/0130635 A1
(43) Pub. Date: Jun. 2, 2011

(54) Medical self-service

(75) Inventor: Gary A. Ross, Dundee (GB)

(73) Assignee: NCR Corporation, Dayton, OH (US)

(21) Appl. No.: 12/592,627

(22) Filed: Nov. 30, 2009

(51) Int. Cl. G06Q 50/00; G06Q 10/00; A61B 5/00; G06F 17/30; G05B 19/00

(52) U.S. Cl. 600/301; 705/3; 600/300; 707/769; 340/5.83; 707/E17.014

(57) ABSTRACT

Techniques for medical self-service are provided. A kiosk interacts with a patient to establish a patient identity, acquire medical records for the patient, and acquire one or more medical vital readings for the patient. The kiosk then packages the information in a summary that is electronically provided to medical personnel before a patient is addressed by the medical personnel at a medical facility.

### BACKGROUND

The dilemma associated with providing medical care is omnipresent in today's economy. Medical care has finite resources and as more and more advances are made, patients desire increasingly more procedures and tests. This is coupled with the fact that in the United States the majority of the population is rapidly approaching retirement age, which is expected to create even more strain on medical care.

By some estimates, health care costs in the United States are approaching 30-40% of the Gross Domestic Product (GDP). Some 20-30% of the GDP is used to service the debt of the United States. Another 10-20% is associated with defense spending. One can readily see that there is little left in the GDP to run the government or provided other needed services to the citizens.

Nearly every politician, health care expert, and economist has provided some piecemeal solutions to the health care problems. However, often because of special interest groups and the lack of will from the citizens to increase taxes in any substantial way, very little has been achieved and the prospects of a solution remain dismal.

One aspect of medical costs is associated with merely processing a patient before a patient is seen. This can be a time consuming processing involving multiple medical staff. A patient has to be identified, insurance has to be established (or lack thereof), medical records or history of the patient have to be acquired, and basic vitals are taken from the patient. All of this occurs with multiple staff and before a physician or nurse ever actually sees the patient. As a result, a medical facility may have 2-3 or even more support staff for each physician or nurse. Moreover, some patients may be in dire need of care and yet in only the most severe cases will a patient bypass all the procedures needed to intake the patient. It is apparent that this entire process is one aspect of medical care that is in significant need of automation.

### SUMMARY

In various embodiments, techniques for medical self-service are presented. According to an embodiment, a method for medical self-service is provided. Specifically, an identity of a medical patient is identified via a biometric sensor. Next, medical data is accessed, over a network, to acquire medical records for the medical patient using the identity. Then, the medical patient interacts with one or more automated sensors to acquire one or more readings for medical vitals being experienced by the medical patient. Finally, the medical records and the readings are automatically assembled into an information packet for medical personnel to address the medical patient.

### BRIEF DESCRIPTION OF THE DRAWINGS

FIG. 1 is a diagram of a method for medical self-service, according to an example embodiment.

FIG. 2 is a diagram of another method for medical self-service, according to an example embodiment.

FIG. 3 is a diagram of a medical self-service system, according to an example embodiment.

### DETAILED DESCRIPTION

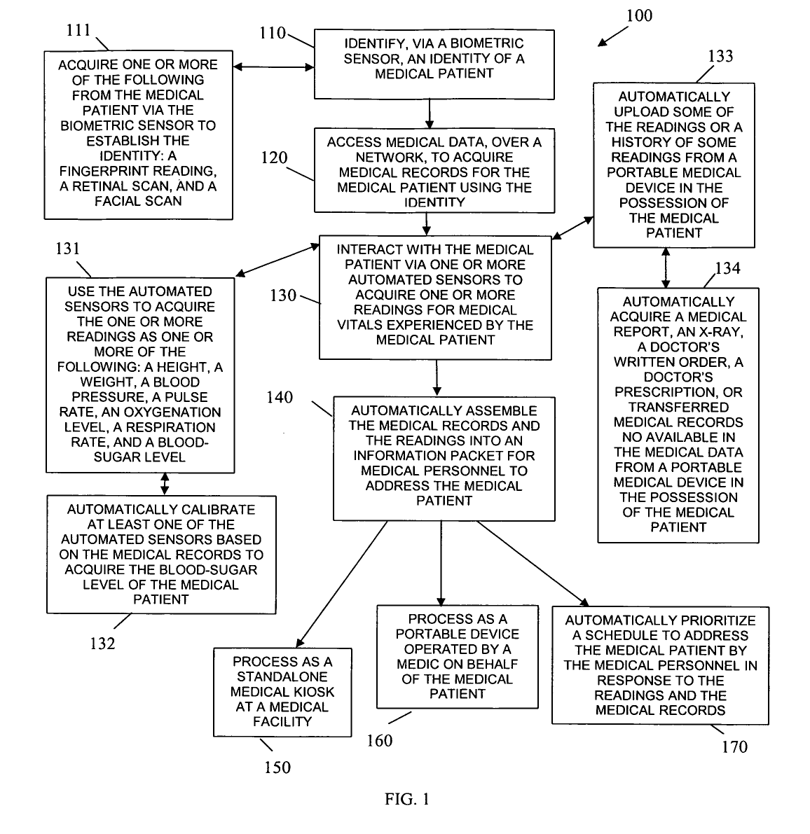

FIG. 1 is a diagram of a method 100 for medical self-service, according to an example embodiment. The method 100 (hereinafter “medical intake service”) is implemented as instructions residing on a computer-readable storage medium and executed by one or more processors. The processors are specifically configured to process the medical intake service. The medical intake service operates over a network. The network is wireless, wired, or a combination of wired and wireless.

At 110, the medical intake service identifies, via a biometric sensor an identity of a patient. The biometric device is a biometric reader integrated into or interfaced to a kiosk or portable device that processes the medical intake service. In some cases, the biometric sensor is separate from the kiosk or device processing the medical intake service and in these configurations the biometric sensor communicates with the kiosk or portable device via a wired or wireless communication channel. The biometric sensor can capture biometric data in a variety of manners.

For example, at 111, the medical intake service acquires from the patient and via the biometric sensor a variety of biometric readings, such as a fingerprint reading, a retinal scan, a facial scan, and the like. It is noted that multiple biometric sensors can be used and multiple different types of biometric readings taken to establish the identity of the patient.

At 120, the medical intake service access medical data, over the network, to acquire medicals for the medical patient using the identity. That is, an external database or plurality of databases is automatically and dynamically accessed once the identity is acquired to obtain medical records that pertain to the medical patient. Encrypted communications and authentication can be used when accessing the network to ensure proper patient privacy and to comply with government regulations. A check can also be made that proper patient authorization to access the record has been granted by the patient, and when authorization has not be granted, the patient may be presented with a release on a display screen associated with the kiosk or portable device to provide authorization. So compliance and security can be enforced when acquiring the medical records of the patient.

At 130, the medical intake service interacts with the medical patient via one or more automated sensors to acquire one or more readings for medical vitals being experienced by the patient. A variety of automated sensors already exists that do not require a separate individual to administer to the patient.

For example, at 131, the medical intake service uses the automated sensors to acquire the one or more readings as one or more of the following: a height, a weight, a blood pressure, a pulse rate, an oxygenation level, a respiration rate, a blood-sugar level, and the like.

Continuing with the embodiment of 121 and at 122, the medical intake service automatically calibrates at least one of the automated sensors based on the medical records to acquire the blood-sugar level of the medical patient. That is, medical records can be used to calibrate some of the sensors that take the medical vital readings from the patient.

In another case, at 133, the medical intake service automatically uploads some of the readings and/or some medical history from a portable medical device in the possession of the medical patient. The portable medical device is designated as any portable device having medical data that can be wirelessly transmitted in an automated fashion to the kiosk or device processing the medical intake service. This can be done via Bluetooth discover, a call, infrared, wireless networking and the like. The device can be a phone, a Personal Digital Assistant (PDA), a laptop, a netbook, a wearable device having medical information, a blood-sugar device, and the like.

Continuing with the embodiment of 133 and at 134, the medical intake service automatically acquires a variety of additional information from the portable device in the possession of the patient, such as: a medical report, an x-ray, a doctor's written order, a doctor's prescription, transferred medical records not available with the medical data, and the like.

At 140, the medical intake service automatically assembles the medical records and the readings into an information packet for the medical personnel to address the medical patient. The types of information or selective portions of the information contained in the information packet can be configured by medical staff or the preferences of a particular physical or policy of a medical facility.

According to an embodiment, at 150, the entire medical intake service is processed on a standalone kiosk having processor, memory, and storage resources.

In another embodiment, at 160, the entire medical intake service is processed as a portable device operated by a medic on site where a patient is being processed.

In another situation, at 170, the medical intake service automatically prioritizes a schedule to address the medical patient by the medical personnel. This is done in response to or based on the readings and the medical records. So, vitals or medical history warranting expedited attention can be handled and prioritized in an automated, real time, and dynamic manner by the medical intake service.

It is also noted that the kiosk or the portable device that processes the medical intake service can interact with the patient or the operator of the device to ask a series of triage questions of the patient to acquire additional information. Answers provided to some questions can drive additional questions to rule out certain diagnoses or confirm other diagnoses. These answers to the questions are incorporated into the information packet delivered to the medical personnel.

Furthermore, the information packet can be delivered to medical personnel electronically and/or sent automatically to a printer for hard copy that is delivered to the doctor before seeing the patient.

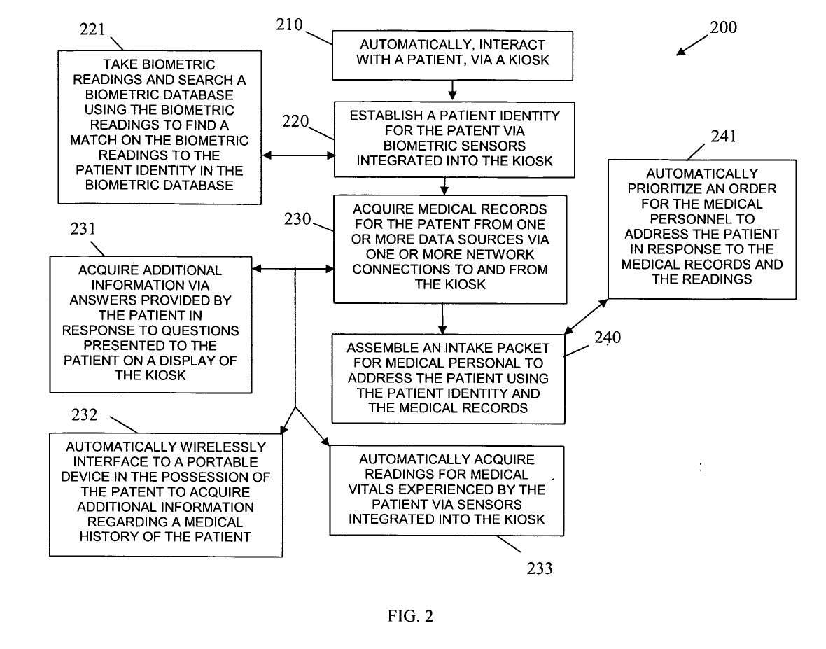

FIG. 2 is a diagram of another method 200 for medical self-service, according to an example embodiment. The method 200 (hereinafter “medical preprocessing service”) is implemented as instruction within a computer-readable storage medium that execute on one or more processors, the processors specifically configured to execute the medical preprocessing service. The medical preprocessing service is operational over a network; the network is wired, wireless, or a combination of wired and wireless.

The medical preprocessing service represents another and in some cases enhanced perspective of the medical intake service, presented above with respect to the discussion of the FIG. 1.

At 210, medical preprocessing service automatically interacts with a patient via a kiosk. The interactions that occur are discussed below with reference to the processing of 220-241.

Specifically, at 220, the medical preprocessing service establishes a patient identity via biometric sensors or readers integrated into the kiosk.

According to an embodiment, at 221, the medical preprocessing service takes biometric readings and searches a biometric database using the biometric readings to find a match on the biometric readings to the patient identity in the biometric database.

At 230, the medical preprocessing service acquires medical records for the patient from one or more data sources via one or more network connections that exist coming into and going out from the kiosk.

In a particular case, at 231, the medical preprocessing service acquires additional information via answers provided by the patient in response to or based on questions presented to the patient on a display of the kiosk.

In another situation, at 232, the medical preprocessing service automatically and wirelessly interfaces to a portable device in the possession of the patient to acquire additional information regarding a medical history or medical readings for the patient.

It is noted that the kiosk may also display screens to the patient that permits the patient to log onto web-based services having medical information for the patient and authorize the transferring of such data to the medical preprocessing service.

In still another scenario, at 233, the medical preprocessing service automatically acquires readings for medical vitals experienced by the patient via sensors integrated into the kiosk.

At 240, the medical preprocessing service assembles an intake packet for medical personnel to address the patient using the patient identity and the medical records. The intake packet sent to medical personnel before the patient is seen.

According to an embodiment, at 241, the medical preprocessing service prioritizes an order for the medical personnel to address the patient in response to or based on the medical records and the readings. So, patients in need of expedited care can receive such care in a timely fashion.

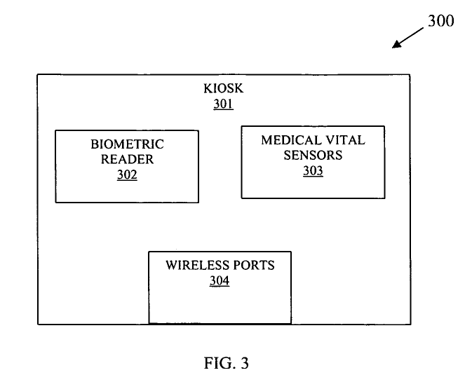

FIG. 3 is a diagram of a medical self-service system 300, according to an example embodiment. The medical self-service system 300 is implemented as instructions residing in computer-readable storage media and to execute on one or more processors of a network.

The medical self-service system 300 implements, inter alia, the methods 100 and 200 of the FIGS. 1 and 2, respectively.

The medical self-service system 300 includes a kiosk 301, a biometric reader 302, and medical device sensors 303. In an embodiment, the medical self-service system 300 also includes one or more wireless ports 304. Each of these and their interactions with one another will now be discussed in turn.

The kiosk 301 includes one or more processors having instructions that execute on the processors to perform such things as the methods 100 and 200 of the FIGS. 1 and 2, respectively. The kiosk 301 can be situated at a medical facility or at a remote location where medical attention is needed, such as a military hospital, a triage center at a disaster site, a developing third-world country refugee or medical camp, and the like. So, the kiosk 301 can be moved to locations where needed.

The kiosk 301 is configured to interact with the biometric reader 302 to acquire biometric readings from a patient that interacts with the kiosk in order to establish a patient identity for the patient.

Additionally, the kiosk 301 is configured to access the network to acquire medical records in response to the patient identity.

Moreover, the kiosk 301 is also configured to interact with the medical vital sensors 303 to acquire vital readings being experienced by the patient. The one or more processors are configured to package the patient identity, the medical records and the vital readings into a summary for electronic delivery over the network to medical personnel before the medical personnel address the patient.

The summary can be integrated into a medical intake processing system, automatically sent to a printer, sent to a computer, sent to a PDA of medical personnel, and the like.

According to an embodiment, the one or more processors are further configured to modify an intake schedule in order to place and prioritize the patient identity within a queue that the medical personnel uses to address the patient and other patients at a medical facility. So, should it be deemed that the medical vital readings or medical records or even systems experienced by the patient warrant accelerated attention, then the patient can be expedited for processing within the medical facility.

In another case, the one or more processors are further configured to contact an insurance carrier system of the network to preauthorize the patient for being addressed by the medical personnel. So, the insurance of the patient can be contacted and payment assured before the patient is addressed.

In still another scenario, the one or more processors are further configured to send an alert to medical staff to dispense medicine or ask a physician for authorization to dispense medicine in response to the vital readings.

The biometric reader 302 is integrated into the kiosk 301. The biometric reader 302 can be a device designed to capture biometric data, such as but not limited to, fingerprint readings, thumb print readings, digit distance readings (distance between digits and lengths of digits (fingers and/or thumbs), retinal scans, facial scans, voice patterns, and the like.

The medical device sensors 303 are also integrated into the kiosk 301. The medical device sensors 303 include a variety of sensors configured to capture readings associated with medical vitals of a patient interacting with the kiosk 301. These readings can include, by way of example only, weight, height, blood pressure, heart rate, respirations, blood sugar level, oxygenation level, and the like.

In some cases, the medical self-service system 300 includes one or more wireless ports 304. The wireless ports 304 are integrated into the kiosk 301 and are configured to wireless upload additional medical information from portable devices of the patient that are integrated into the summary. These portable devices can include a variety of devices, such as memory sticks, bracelets having memory, personal digital assistants (PDA's), necklaces or other wearable items having memory, phones, and the like. The additional medical information can include, by way of example only, transferred medical records from a physical or another medical facility, x-rays, MRI's, cat scans, sonograms, doctor reports, doctor orders, prescription information, and the like.

It is now appreciated how the medical intake process can be automated to eliminate human resources, reduce errors, increase accuracy, and improve the initial medical intake procedures before a patient is visited or addressed by a physician or a nurse. The techniques presented herein also improve the information received by the physician or nurse before the patient is seen.

The above description is illustrative, and not restrictive. Many other embodiments will be apparent to those of skill in the art upon reviewing the above description. The scope of embodiments should therefore be determined with reference to the appended claims, along with the full scope of equivalents to which such claims are entitled.

The Abstract is provided to comply with 37 C.F.R. §1.72(b) and will allow the reader to quickly ascertain the nature and gist of the technical disclosure. It is submitted with the understanding that it will not be used to interpret or limit the scope or meaning of the claims.

In the foregoing description of the embodiments, various features are grouped together in a single embodiment for the purpose of streamlining the disclosure. This method of disclosure is not to be interpreted as reflecting that the claimed embodiments have more features than are expressly recited in each claim. Rather, as the following claims reflect, inventive subject matter lies in less than all features of a single disclosed embodiment. Thus the following claims are hereby incorporated into the Description of the Embodiments, with each claim standing on its own as a separate exemplary embodiment.

1. A processor-implemented method to execute on a processor configured to execute the method, comprising:
identifying, via a biometric sensor, an identity of a medical patient;
accessing medical data, over a network, to acquire medical records for the medical patient using the identity;
interacting with the medical patient via one or more automated sensors to acquire one or more readings for medical vitals experienced by the medical patient; and
automatically assembling the medical records and the readings into an information packet for medical personnel to address the medical patient.

2. The method of claim 1, wherein identifying further includes acquiring one or more of the following from the medical patient via the biometric sensor to establish the identity: a fingerprint reading, a retinal scan, and a facial scan.

3. The method of claim 1, wherein interacting further includes using the automated sensors to acquire the one or more readings as one or more of the following: a height, a weight, a blood pressure, a pulse rate, an oxygenation level, a respiration rate, and a blood-sugar level.

4. The method of claim 3, wherein using further includes automatically calibrating at least one of the automated sensors based on the medical records to acquire the blood-sugar level of the medical patient.

5. The method of claim 1, wherein interacting further includes automatically uploading some of the readings or a history of some readings from a portable medical device in the possession of the medical patient.

6. The method of claim 5, wherein automatically uploading further includes automatically acquiring a medical report, an x-ray, a doctor's written order, a doctor's prescription, or transferred medical records no available in the medical data from a portable medical device in the possession of the medical patient.

7. The method of claim 1 further comprising, processing the method as a standalone medical kiosk at a medical facility.

8. The method of claim 1 further comprising, processing the method as a portable device operated by a medic on behalf of the medical patient.

9. The method of claim 1 further comprising, automatically prioritizing a schedule to address the medical patient by the medical personnel in response to the readings and the medical records.

10. A processor-implemented method to execute on a processor configured to execute the method, the method, comprising:
automatically, interacting with a patient, via a kiosk having the processor, the interaction including:
establishing a patient identity for the patent via biometric sensors integrated into the kiosk;
acquiring medical records for the patent from one or more data sources via one or more network connections to and from the kiosk; and
assembling an intake packet for medical personal to address the patient using the patient identity and the medical records.

11. The method of claim 10, wherein establishing further includes taking biometric readings and searching a biometric database using the biometric readings to find a match on the biometric readings to the patient identity in the biometric database.

12. The method of claim 10, wherein acquiring further includes acquiring additional information via answers provided by the patient in response to questions presented to the patient on a display of the kiosk.

13. The method of claim 10, wherein acquiring further includes automatically wirelessly interfacing to a portable device in the possession of the patent to acquire additional information regarding a medical history of the patient.

14. The method of claim 10, wherein acquiring further includes automatically acquiring readings for medical vitals experienced by the patient via sensors integrated into the kiosk.

15. The method of claim 14, wherein assembling further includes automatically prioritizing an order for the medical personnel to address the patient in response to the medical records and the readings.

16. A processor implemented system, comprising:
a kiosk having one or more processors and interfaced to a network;
a biometric reader integrated into the kiosk; and
medical vital sensors integrated into the kiosk;
the kiosk is configured to interact with the biometric reader to acquire biometric readings from a patient that interacts with the kiosk to establish a patient identity for the patient, and the kiosk is configured to access the network to acquire medical records in response to the patient identity, and the kiosk is further configured to interact with the medical vital sensors to acquire vital readings being experienced by the patient, the one or more processors configured to package the patient identity, the medical records and the vital readings in a summary for electronic delivery over the network to medical personnel before the medical personnel address the patient.

17. The system of claim 16, wherein the one or more processors are further configured to modify an intake schedule to place the patient identity in a queue that the medical personnel uses to address the patient and other patients at a medical facility.

18. The system of claim 16, wherein the one or more processors are further configured to contact an insurance carrier system of the network to preauthorize the patient for being addressed by the medical personnel.

19. The system of claim 16, wherein the one or more processors are further configured to send an alter to medical staff to dispense medicine or ask a physician for authorization to dispense medicine in response to the vital readings.

20. The system of claim 16 further comprising, one or more wireless ports integrated into the kiosk and configured to wireless upload additional medical information from portable devices of the patient that are integrated into the summary.
